(19) United States
(12) Patent Application Publication
(10) Pub. No.: US 2002/0048598 A1
(43) Pub. Date: Apr. 25, 2002

(54) INTERNALLY SUPPORTED LIPID VESICLE SYSTEMS

(76) Inventor: NAVID MALIK, LONDON (GB)

Correspondence Address: YOUNG & THOMPSON, 745 SOUTH 23RD STREET 2ND FLOOR, ARLINGTON, VA, 22202

(21) Appl. No.: 09/445,767

(22) PCT Filed: Jun. 11, 1998
(86) PCT No.: PCT/GB98/01706

(30) Foreign Application Priority Data
Jun. 13, 1997 (GB) 9712329.3
Mar. 19, 1998 (GB) 9805922.3

(51) Int. Cl. A61K 9/127

(52) U.S. Cl. 424/450

(57) ABSTRACT

A system comprising a branched polymeric structure which provides a structural support for a mono-layer, bi-layer or multi-layered lipid coating. The branched polymeric structure may include dendrimers, arborol or, star polymers, hyperbranched structures, and cascade polymer systems. A method of producing the system is also disclosed. The system is essentially comprised of a structurally supportive core overlaid with a lipid portion. The supportive core may also interact with a biologically active molecule. The core may provide a matrix-like structure, which functions both as a structural support for the lipid portion and a site for interaction with the lipid portion.

The present invention relates to the simulation of a cytoskeleton (artificial cytoskeleton (AS in further text)) for the support of a lipid layer or multi lipid layer coating. The AS can be a branched polymer, cascade polymer, hyperbranched polymer, dendrimer, arborol, tubular polymer or polymeric aggregate or porous micro- or nano-particle (these structures can be synthetic or natural). The coating utilised can be an anionic-, cationic-, or neutral phospholipid (esters of glycerol), sphingomyelin or any other ester of glycerol or sphingol, cholesterol, lipoproteins, glycolipids, or even a reconstituted membrane of animal or plant cell, reconstituted bacterial membrane or viral capsid. The surface of the AS can be charged (e.g. anionic or cationic) or neutral. Examples of possible surface groups of the AS could be NH₂, COOH, CO (keto), CHO (aldehyde), SH, CN, OH, PO₃OH₂, SO₃H, halides, chlorides, iodides, fluorides and other such chemical groups.

The current strategy for the delivery of substances within a biological system is complicated, and poses a major obstacle for the delivery of therapeutic or desired substances. These substances may often have poor water solubility, poor stability in biological fluids, cause an immunogenic or antigenic response or other adverse side reaction, and may have toxicological side effects. They often do not have specificity or targeting, or unfavourable pharmacokinetics. In order to exploit the system presented here, these substances could be either linked to the surface through charge, covalent bond, ionic or weaker bond (e.g. hydrogen, hydrophobic interaction or co-ordinate complexation) or they could be entrapped within the core of this AS or a combination of both. The coating could afford a protection to the contents in the AS and be released either passively or triggered in some way (e.g. by an alteration of pH, temperature, exposure to electromagnetic radiation (light, radio, infra-red, ultra-violet etc.) or mechanical waves, or activity of an enzyme) at any time.

In essence what is being revealed here is the preparation of system which simulates a living animal, plant, bacterial cell or virus. Such a system would therefore differ markedly from other similar systems (e.g. a liposome) because it has a stable or structurally controlled interior support. I would like to call this invention the Articell™.

The Articell is essentially comprised of a structurally supportive core overlaid with a lipid portion. We prefer that the support is a ‘tree-like’ multiply branched or hyperbranched polymer, preferably a carbon based polymer, capable of presenting multiple interaction sites to at least the lipid portion. We prefer that the supportive core can also interact with a biologically active molecule. We prefer that the core provides a matrix-like structure, which functions both as a structural support for the lipid portion and a site for interaction with the lipid portion.

Medicine has failed in the treatment of many diseases in some cases chronic treatment still seems the only alternative to finding a “cure”. Just as viruses have found ways of exploiting the biological environment to replicate and multiply, so there is a growing need to compete at the molecular level to overcome the existing problems. The Articell™ will overcome these problems because among its other strengths it will appear as a normal cell to the host; and yet its payload (the contents contained within the coating or attached to its surface) could be tailor made to fit any desired requirement. Several different compounds could be trapped within the Articell™ or exposed on its surface and each component could be released in a predefined way at a desired site by including targeting moieties at the surface. Essentially the Articell™ will act as a biological cell.

In the treatment of Cancer or viral diseases, there are currently problems associated with non specific toxicity of drugs used in therapy. Often they never reach their intended site of action because of their poor water solubility or rapid elimination from the host. Water insoluble substances may require toxic or otherwise unsuitable vehicles for administration. They may be unstable in biological fluids or/are rapidly excreted or metabolised. Enclosure within the Articell™ or conjugation to its surface could eliminate such problems, by increasing water solubility, or increase their stability and half life by preventing their degradation, modification or excretion whilst enclosed or attached to the surface. Whilst enclosed or attached to the surface of the Articell™ this should lead to reduced toxicity of the substance.

In this respect I have suggested the following possible treatments but this list is by no means exhaustive and is only intended as a guide. Essentially in a broader sense I am revealing the “hardware” needed for the simulation and creation of a “living” cell, which could incorporate specific cellular compartments.

### Delivery routes

The Articell™ could be delivered via the following routes: Oral, nasal, intravenous (i.v.), intraperitoneal (i.p.), subcutaneous (s.c.), intramuscularly (i.m.), transdermal, or any other traditionally used delivery route.

Examples of uses

1. Coating the surface with specific receptors could allow the Articell™ to “mop up” bacteria, toxins or viruses in the circulation of a host before being excreted or otherwise degraded.

2. Enclosure of nano-machines (mechanical/electronic) could interface the engineering and biological worlds. Nano-machines which could perform simple or complex tasks could be enclosed in the Articell™ and released at a specific target site.

3. Single or multiple vaccinations on the same system

Examples of treatments of the following disease families

1. Allergies (e.g. Hay fever)

2. Viral (e.g. AIDS)

3. Bacterial

4. Cancer

5. Cardiovascular disorders

6. Hormonal (i.e. diabetes)

7. Inflammation

8. Protozoal

9. Toxin contamination

A variety of systems have been explored as potential drug delivery applications, each has a varied level of success but also significant drawbacks and problems which have prevented them from wider and more successful use. In addition there are fundamental problems still facing such therapies which will not easily be overcome either in the present or the future. These problems could revolve around stability, size, solubility, toxicity or characterisation of end products. The current and state of the art systems are given below.

Low molecular weight prodrugs (4), Macromolecular carriers (including immunoconjugates (5), natural polymers (6), synthetic polymers (7), vesicular or particulate systems (liposomes (8, 9), nanoparticles (10), microparticles for regional therapy ( 11)) or polymeric implants (12, 13). Most of these approaches are based on combinations of drug with polymer. The polymer serves as a carrier system wherein the drug is dispersed or dissolved, or to which it is covalently linked.

Examples of these problems are listed below for some of these systems:

1. Soluble Polymers

Polydispersity of molecular weights through difficulties in synthesis, lead to a broad dispersity of end product i.e. conjugate with the drug. Problems in characterisation of polymer-adducts, and difficulty in determination of the exact composition. The polydispersity complicates the pharmacokinetics of drug release or action and leads to unpredictable therapeutic effects. Large wastage of compound at each step of synthesis (low yields) and final compound once administered because of rapid excretion or narrow therapeutic index.

2. Monoclonal antibodies

High molecular weight, immunogenicity and antigenicity and rapid biodegradation. Complicated conjugation chemistry. Therapeutic compound is often taken away from intended site of action. Large wastage of compound at each step of synthesis (low yields) and final compound once administered because of rapid excretion or narrow therapeutic index.

3. Microspheres and nanospheres

Large porous materials that leak their contents indiscriminately. Microspheres are eliminated rapidly by the reticulo endothelial system (RES) of the host, and have undesirable accumulation in the host. Both show undesirable toxic effects.

4. Retroviruses

Dangers of host genome incorporation and uncontrolled replication. Can be immunogenic, complicated and expensive to prepare.

5. Liposomes

Large size and lack of stability of system, leading to leakage of contents, lipid layer prone to disintegration and consequential toxicity, immunogenicity and antigenicity. Rapidly removed by RES.

It is a particular object of the invention to alleviate the aforementioned problems in relation to liposome systems.

Example of the preparation of an Articell™

Here I propose the coating of a dendrimerA with a phospholipid bilayer as an example of the preparation of Articell™

### EXAMPLE 1

Dendrimers of X generation with positively charged surface groups and anionic phospholipids are mixed in organic solvent. After evaporation of solvent, the mixture of dendrimers and phospholipids is resuspended in water or aqueous buffer, dialysed and freeze dried. Solid substance will contain the purified Articell™.

### EXAMPLE 2

In a similar way a single layer of lipids containing COOH as a reactive group could be covalently linked to the surface of the dendrimer containing NH₂ as the reactive group. In a second step further layers of lipids (polar or non polar) are added to create further layers on the dendrimer, in a suitable solvent. The Articell™ is then isolated and purified in a similar way to example 1.

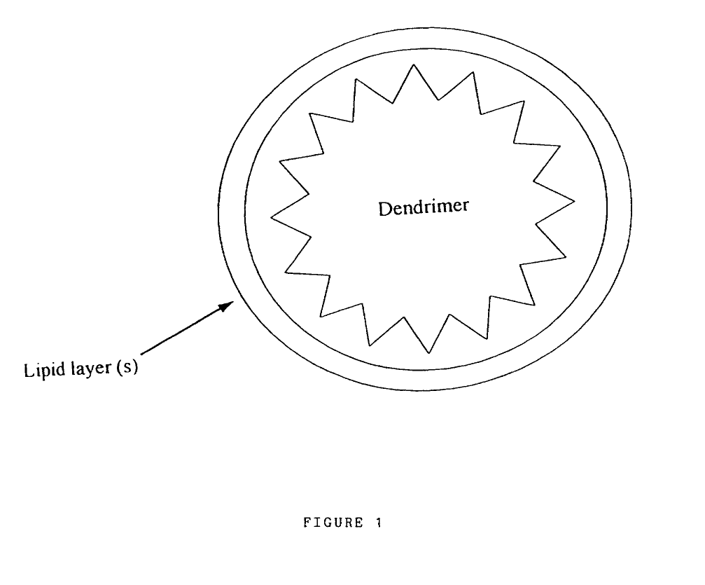

FIG. 1 Shows a scematic example of an Articell™ in accordance with the invention.

In each of the examples above the lipid layer is supported in a stable manner. A Dendrimers (1, 2, 3) are branched polymers consisting of generations. They can be produced in successive generations each with a defined size, number of external functional groups and molecular weight. As the generation size increases the molecular weight and no. of functional groups approximately doubles. A dendrimer consists of a core, an internal unit and a terminal unit. The core of the dendrimer can vary quite markedly, including the repeating internal unit and the terminal unit and so far 150 families of dendrimer have been synthesised or proposed.

Characterisation

Characterisation can be made using chemical, physical, biochemical or biological methodologies. Physical strategies include different chromatographic methods e.g. thin layer chromatography (TLC), high performance liquid chromatography (HPLC). Spectrometry such as ultraviolet-visible, infrared, mass spectrometry. Circular dichroism (CD), atomic absorption spectroscopy (AAS), nuclear magnetic resonance (NMR) spectroscopy, viscometry, refractometry, differential scanning calorimetry (DSC), X-ray crystallography, tunnelling and force field microscopy.

In all kits the preparation and characterisation can be achieved easily using conventional methods. The scale up technology is in place and is relatively inexpensive. The raw materials are readily available.

The following prior art is hereby acknowledged:

1. E. Buhleier, W. Wehner, F. Vogtle, Synthesis, 1978, 155.

2. P. J. Flory, J. Am. Chem. Soc. 1952, 74, 2718.

3. Tomalia, D. A., Baker, H., Dewald, J. R., Hall, M., Gallos, G., Martin, S., Roeck, J., Ryder, J., Smith, P., (1985) Polym, J. 17, 117.

4. Waller, D. G. and George, C. F. (1989) Prodrugs. Br. J. Clin. Pharmacol. 28, 497-507.

5. Baldwin, R. W. Byers V. S. and Mann, R. D. (Eds) (1990) In: Monoclonal antibodies and immunoconjugates. Parthenon Publishing, Carnforth.

6. Sezaki, H., Takakura, Y. and Hashida, M. (1989) Soluble macromolecular carriers for delivery of antitumour agents. Adv. Drug. Rev. 3, 247-266.

7. Putnam, D. and Kopocek, J. (1985) Polymer conjugates with antitumour activity. Adv. Polym. Sci. 122, 55-123.

8. Rahman, A. and Schein, P. S. (1988). Use of liposomes in cancer chemotherapy. In: G. Gregoriadis (Ed.), Liposomes as drug carriers. John Willey, New York, PP. 381-400.

9. Gabizon, A. (1989). Liposomes as a drug delivery system in cancer chemotherapy. In: F. H. D. Roerdink and A. M. Kroon (Eds), Drug carrier systems. Vol. 9. John Wiley, New York, pp. 185-212.

10. Brannonpeppas, L. (1985). Recent advances on the use of biodegradable microparticles and nanoparticles in controlled drug delivery. Int. J. Pharm. 116. 1-9.

11. Kerr, D. J. and Kaye, S. B. (1991) Chemoembolism in cancer chemotherapy. CRC Crit. Rev. Ther. Drug. Carrier Sys. 8, 19-39.

12. Vansavage, G. and Rhodes, C. T. (1995). The sustained release coating of solid dosage forms: a historical review. Drug Dev. Indust. Pharm. 21, 93-118.

13. Yang, M. B. Tamargo, R. J. and Brem, H. (1989). Controlled delivery of 1,3-bis(2-chloroethyl)-1-nitrosourea from ethyl -vinyl acetate copolymer. Cancer Res. 49, 5103-5107.

14. Hawker, C. and Frechet J. M. J (1990). J. Chem Soc., Chem Commun. 1010.

15. de Brabander-van der Berg EMM and Meijer E. W. (1993). Angew Chem Int Ed Engl. 105, 1370-1373.

16. Roy, R (1996). Glycodendrimers: a novel biopolymer. Polymer news. 21, 226-232.

### STATEMENT OF NOVELTY

The invention provides in one aspect the system comprising a branched polymeric structure which provides a structural support for a mono-layer, bi-layer or multi-layered lipid coating.

The invention provides in another aspect the system where the synthesis of the support could be initiated within a coating that has already been pre-formed e.g. phospholipid or cholesterol layer(s) forming a vesicle or liposomal structure. So that the structural support evolves or grows within the coating until its completion. The final structure being the support contained within the coating.

The invention provides yet another aspect of the system where the use of the Articell™ is for the purposes of drug delivery for disease or medical use or as an imaging agent or diagnostic for a disease or medical use.

The invention also provides a method for the production of a system according to the invention, wherein a dendrimer, arborol, star polymer, hyperbranched structure, cascade polymer or fragment thereof, such as a dendrimer branch or fragment synthesised by a convergent route, is assembled into a micelle structure, such as by the attachment of a hydrophobic coating at one end, in an aqueous solvent, such as water, and then a lipid coating is applied.

It is preferable that at least one of the structural support and the lipid coating are water soluble.

Step 1: Synthesis of the Internal Support

1. Dendrimer (Cascade Polymer, Hyperbranched Polymer, Arborol)

The methods described for the synthesis of dendrimers have been previously described in the literature.

Dendrimers possess three structural features, which afford them their unique and distinctive properties (structural or otherwise). They have an initiator core, interior areas, which have cascading tiers or branch cells with radial connectivity to the initiator core and an exterior or surface region of terminal moieties attached to the outermost generation.

Two general methods have been proposed to synthesise a dendrimer. The divergent route where synthesis begins from the core, or the convergent route where synthesis begins from the terminal groups. In addition, one step synthesis can be employed or multi-step in the formation of the dendritic structure.

Divergent dendritic construction results from sequential monomer addition beginning from a core and proceeding outward toward the macromolecular surface. To a respective core representing the zeroth generation and possessing one or more reactive site(s), a generation or layer of monomeric building blocks is covalently connected. The number of building blocks that can be added will be dependent on the number of available reactive sites on the particular core assuming parameters, such as monomer functional group steric hindrance and core reactive site accessibility, are generally not a concern. Repetitive addition of similar, or for that matter dissimilar, building blocks (usually effected by a protection-deprotection scheme) affords successive generations. A key feature of the divergent method is the exponentially increasing number of reactions that are required for the attachment of each subsequent tier (layer or generation).

The convergent dendritic construction is a strategy whereby branched polymeric arms (dendrons) are synthesised from the “outside-in”. This concept can be best described by envisioning the attachment of two terminal units containing a reactive group to one monomer possessing a protected functionality, resulting in the preparation of the first generation or tier. Transformation of the active or focal site followed by treatment with 0.5 equivalent of the masked monomer affords the next higher generation.

One-step hyperbranched polymers are synthesised by direct a one-step polycondensation of AₓB monomers, where x equal or greater than 2. Graft-on-graft procedure (chloromethylation followed by anionic grafting) has been used to synthesise tree-like structures.

At least 150 families of dendrimers have been synthesised and recorded in the literature over the past decade or so. In this respect it is impossible to describe every possible method of synthesis. Many more dendrimers are becoming commercially available.

Because of the large number of possibilities of synthesis only the two main routes will be described here, both methods have been described in the literature.

### SYNTHETIC METHODOLOGIES

i) Divergent Procedures

### EXAMPLE 1

Synthesis of Polyamidoamine Dendrimers (Tomalia et. at. 1985)

Synthesis is by an alternating sequential reaction using ethylene diamine (H₂N—CH₂—CH₂—NH₂), and via Michael's addition, reacting methyl acrylate (H₂C═CH—COOCH₃) to produce a methyl ester (half generation, carboxy terminated), further addition with ethylene diamine produces the full generation (amine terminated) and extension of the dendritic branching. A purification step is incorporated into the reaction to achieve selectivity for size. The chemistry is shown schematically below:

[chemical structure]

As the reaction proceeds the number of functional groups at the terminus is doubled. Successive generations or half generations are synthesised by repeating the steps with an excess of the monomer, and incorporating a purification and characterisation step at each stage of synthesis.

### EXAMPLE 2

Synthesis of Nitrile and Carboxylate Terminated Dendrimers (Meijer et. al, 1993)

The synthesis of poly(propylene imine) dendrimers from a diaminobutane core were made by Michael's addition of acrylonitrile to primary amines, followed by heterogeneously catalysed hydrogenation of the nitrites, resulting in a doubling of the number of primary arnines. 1,4-diaminobutane was used as a core; a number of molecules with either primary or secondary amine groups can also be used. All Michael's reactions were performed using 2.5-4.5 equivalents of acrylonitrile per primary amine at a concentration of 0.1M in aqueous solution. The first equivalent of acrylonitrile was added at room temperature and the second equivalent at 80° C. The reaction time for the complete conversion increased with every generation: 1 h for generation 0.5 (DAB-dendr-(CN)₄), 3 h for generation 4.5 (DAB-dendr-(CN)₆₄). The excess of acrylonitrile was distilled off as a water azetrope. A two-phase clear system was left which allowed the isolation of pure dendrimers with nitrile terminations by pouring off the water layer. Impurities (monomer) were removed by washing residue with distilled water. Hydrogenations of cyanoethylated structures with H₂ (30-75 bar) and Raney/Cobalt as a catalyst were carried out in water. The reaction time was monitored and increased with generations. Amine (NH₂) terminated dendrimers were isolated by evaporating the water from the filtered reaction mixture. Carboxylate terminated dendrimers were obtained by saponification of the nitrile dendrimer, by dissolving them in HCL (˜40%) and refluxing for 2 h. The dendrimers were then precipitated to yield the carboxylic acid terminated dendrimer.

(DAB-dendr-(CN)ₓ—DiAminoButane core dendrimer with x nitrile end groups)

### EXAMPLE 3

Synthesis of N-Chloroacetylated Dendrimers (from Roy et. al, 1996)

Dendrimers were synthesised by solid phase peptide chemistry using 9-fluorenylmethoxycarbonyl (Fmoc) amino-protecting groups and benzotriazolyl esters as the coupling agents. The core used was L-lysine, to which the layers or generations were built. The advantage of this approach to synthesis is the higher yields and well established peptide chemistry.

Dendritic L-lysine cores were elaborated with p-benzyloxybenzyl alcohol (Wang) resin 0.58 or 0.6 mmol/g) to which was anchored a b-alanyl spacer using the previous Fmoc/benzotriazolyl ester strategy (Fmoc-b-Ala-OBt, 2 or 3 equiv., 0.5 equiv. DMAP, DMF, 2.5 or 3 hr). N^{a}, N^{e}-Di-Fmoc-L-lysine were synthesised in approx. 70% yield using well established procedure with 9-fluorenylmethyl chloroformate in 10% sodium bicarbonate. The corresponding benzotriazolyl ester derivative was freshly prepared in N,N-dimethylformamide (DMF) with one equivalent each of N-hydroxybenzotriazole (HOBt) and diisopropylcarbodiimide (DIC, 0° C., then 25° C. for 1 hr). In each cycle, the Fmoc-protecting groups were removed by b-elimination process using 20-25% piperidine in DMF. The degree of coupling was established spectrophotometrically by quantitation of the released dibenzofulvene chromophore at 300 nm following the piperidine treatment.

The products resulting from each sequential generation were then directly treated with pre-formed chloroacetylglycylglycine benzotriazolyl ester prepared by the above procedure. The chloroacetylglycylglycine is commercially available and did not require individual couplings of glycine residues and capping with chloroacetic anhydride as is commonly done. The completion of full derivatisation was determined by the ninhydrin test.

The ninhydrin test is used for the detection of amine groups (e.g. primary) and firstly involves the-preparation of ninhydrin (using buffer, DMSO, hydridantin and ninhydrin; available as a commercial reagent), incubation at 70° C. with the amine groups to be detected and quantification by colorimetric changes spectrophotometrically (570 nm). A standard calibration curve is also constructed using an amino acid such as phenyl-1-alanine. The assay is sensitive to the nano-molar range.

Using the solid phase approach, di-, tetra-, octa-, and hexadeca-valent chloroacetylated dendrimers were obtained in the first, second, third and fourth generations respectively. Structural and purity determinations were assessed by releasing the corresponding unbound chloroacetylated acid derivatives from the polymer support by treatment with aqueous trifluoroacetic acid (95% TFA, 1.5 hr). Dendrimers with yields of >90% were obtained with purity between 90-95%.

While still attached to the resin, each dendrimer generation was treated with an excess of 2-thiosialic acid derivative (1% triethylamine/DMF, 16 hr, 25° C.). The dendrimers were analysed using ¹H-NMR and ¹³C-NMR.

Examples of other branch synthetic methodologies that can be used for synthesis of dendrimers by the divergent route:

1→2 N-Branched

1→2 N-Branched and Connectivity

1→2 N-Branched, Amide Connectivity

1→2 Aryl-Branched, Amide Connectivity

1→2 Aryl-Branched, Ester Connectivity

1→2 C-Branched

1→2 C-Branched, Amide Connectivity

1→2 C-Branched and Connectivity

1→2 C & Aryl-Branched and Connectivity

1→2 Aryl-Branched, N-Connectivity

1→2 Ethano-Branched, Ether Connectivity

1→2 Si-Branched and Connectivity

1→2 P-Branched and Connectivity

1→3 C-Branched

1→3 C-Branched, Amide Connectivity

1→3 C-Branched, Amide (‘Tris’) Connectivity

1→3 (1→2) C-Branched, Amide Connectivity

1→3 C-Branched, Amide (‘Bishomotris’) Connectivity

1→3 C-Branched, Amide (‘Behera's Amine’) Connectivity

1→3 C-Branched and Connectivity

1→3 C-Branched, Ether Connectivity

1→3 C-Branched, Ether & Amide Connectivity

1→3 N-Branched and Connectivity

1→3 P-Branched and Connectivity

1→3 Si-Branched and Connectivity

1→3 Adamantane-Branched, Ester Connectivity

ii) Convergent procedure

### EXAMPLE 4

Synthesis of Polyether Dendrimers (Frechet et at, 1990)

An example of the synthesis of the dendrimer by the convergent approach can be made by the synthesis of a family of dendritic polyether macromolecules based on 3,5-dihydroxybenzyl alcohol 1 as the monomer unit. This monomer can give rise to very high yields from the formation of benzyla ethers from phenols and benzylic halides. In the example the various generation dendritic molecules will be designated by use of the following notation [G-x]-f in which [G-x] refers to the generation number (x=O, 1, 2, . . .) and f refers to the functional group located at the focal point. After coupling to the core, the notation [G-x]ₙ-[C] will be used where n represents the number of dendritic fragments (generation x) coupled to the core. Starting from the benzylic bromide 2, which is the first generation benzylic bromide [G-1]-Br, the reaction can be examined in a variety of solvents (DMF, 1,4-dioxane, THF, acetone, 3-methylbutan-2-one) and a variety of bases (Cs₂CO₃, KOH, K₂CO₃) in the presence or absence of phase-transfer agents. The optimum conditions in terms of yield and synthetic ease have been found to include the use of potassium carbonate and 18-crown-6 in refluxing acetone under vigorous stirring for 48 h. It is essential to maintain efficient stirring throughout the reaction in order to maintain a high rate of conversion. Reaction of 2 and 1 give second-generation benzylic alcohol [G-2]-OH, which can be isolated in ˜90% yield after recystallisation. The C-alkylation has been observed as a crude reaction product by high-field ¹H and ¹³NMR spectra. Similarly, no C-alkylation is detected in latter generations. The reaction of [G-2]-OH with 1 gives the next-generation alcohol [G-3]OH 3 in -88% yield after purification by flash chromatography. In this case, as with subsequent generations, it has been found that reaction with PBr₃ leads to lower yields when compared to brominations with CBr₄/PPh₃. Having obtained the third-generation bromide [G-3]-Br by reaction with 3 with CBr₄/PPh₃, it is possible to proceed to generation 4. Subsequent reactions for generation 4 lead to the higher generation's up to generations 5 and 6. After high purification of the dendritic wedges has been obtained, coupling to a polyflnctional core can be carried out. The polyfunctional core is then chosen and in this example could be 1,1,1-tris (4′-hydroxyphenyl) ethane ([C]-(OH)₃). The dendritic wedges are then brought together to make the dendrimer.

### EXAMPLE 5

Convergent Synthesis of Carbohydrate Dendrimers (from Stoddart et al, 1997)

Tris(hydroxymethyl)methylamine (TRIS) was used as the starting material., onto which three carbohydrate units were located. Glucose was used as a source of the glycosyl donors towards the hydroxymethyl groups in TRIS and therefore as the carbohydrate residue present as the outer-generation of the dendrimers. The free amino group in TRIS, after glycosylation, enables further elaboration through the formation of amide bonds with either branch-point synthons or, where steric problems exist, with spacer synthons possessing appropriate carboxyl functionalities. Amine functionalities are required for the branch-point and spacer synthons. Glycine (amino acetic acid) and 3,3′-iminodipropionic acid were chosen as sources of spacers and interior branch residues. Upon completion of the synthesis of the saccharide-containing dendrons, the final step was attachment of the dendrons to a multi-podent core. A 1,3,5-benzenetricarbonyl-derived unit was selected in order to provide the final dendrimer with a triply branched core.

Examples of other branch synthetic methodologies that can be used for synthesis of dendrimers by the convergent route:

1→2 C-Branched

1→2 C-Branched and Connectivity

1→2 C-Branched, Ether Connectivity

1→2 C-Branched, Ether Connectivity

1→2 Ethano-Branched, Ether Connectivity

1→2 Aryl-Branched

1→2 Aryl-Branched and Connectivity

1→2 Aryl-Branched, Ether Connectivity

1→2 Aryl-Branched, Amide Connectivity

1→2 Aryl-Branched, Ether and Amide Connectivity

1→2 Aryl-Branched, Ether and Urethane Connectivity

1→2 Aryl-Branched, Ester Connectivity

1→2 Aryl-Branched, Ether and Ester Connectivity

1→2 Aryl-Branched, Ether and Ketone Connectivity

1→2 Aryl-Branched, Ethyne Connectivity

1→2 N-Branched

1→2 N-Branched, Amide Connectivity

1→2 C- & N-Branched, Ester Connectivity

1→2 Si-Branched, Silyloxy Connectivity

iii) One-step (hyperbranched) procedures

1→2 Aryl-Branched

1→2 Aryl-Branched and Connectivity

1→2 Aryl-Branched, Ester Connectivity

1→2 Aryl-Branched, Ether Cornectivity

1→2 Aryl-Branched, Ether and Ketone Connectivity

1→2 Aryl-Branched, Amide Connectivity

1→2 Aryl-Branched, Carbamate Connectivity

1→2 Aryl-Branched, Urethane Connectivity

1→2 Aryl-Branched, Ether and Ester Connectivity

1→2 C-Branched

1→2 C-Branched, Ester Connectivity

1→2 C-Branched, Ether Connectivity

1→2 C-Branched, Amide Connectivity

1→2 Aryl-Branched, C-Connectivity

1→2 N-Branched and Connectivity

1→3 Ge-Branched and Connectivity

1→3 (2) Si-Branched and Connectivity

iv) Chiral Dendritic Macromolecules (Divergent Procedures to Chiral Dendrimers)

1→3 C-Branched, Ether and Amide Connectivity

1→2 C-Branched

1→2 Aryl-Branched, Ester and Amide Connectivity

1→2 Aryl-Branched, Ether and Ester Connectivity

1→2 N-Branched and Connectivity

1→2 N-Branched and Connectivity

1→2 N-Branched, Amide-Connectivity

iv) Chiral Dendritic Macromolecules (Convergent Procedures to Chiral Dendrimers)

1→2 Aryl-Branched, Ether Connectivity

1→2 C-Branched, Amide Connectivity

1→2 Aryl-Branched, Ether Connectivity

1→3 P- and Aryl-Branched, P- and Ether-Connectivity

2. Nano-particle

Principle Methods of Preparation:

2A. In Situ Polymerisation

2.A. 1. Nanospheres

a. Emulsification polymerisation in an aqueous or in organic phase.

b. Dispersion polymerisation in an aqueous phase.

2.A.2. Nanocapsules

a. Interficial polymerisation

b. Interficial polycondensation using electrocapillarity emulsification.

2.B. Dispersion of a pre-formed polymer

2.B. 1. Nanospheres prepared from natural macromolecules

a. Emulsification-based methods

b. Phase separation-based methods

2.B.2. Nanospheres prepared from synthetic polymers

a. Emulsification-based methods

1. Emulsification-solvent extraction

2. Salting-out

3. Emulsification-diffusion

b. Direct precipitation-based methods

2.B.3. Nanocapsules prepared by interficial deposition of a synthetic polymer.

### EXAMPLE 6

Synthesis of Nanoparticle (PLGA)

The emulsification-solvent evaporation method was used to prepare monensin nanoparticles using biodegradable PLGA polymer. Initially 200 mg of copolymer PLGA and 20 mg of monensin were dissolved in 25 ml acetone. Two hundred mg of polyvinyl alcohol was dissolved in 50 ml distilled water. The polymer solution containing monensin was added to the aqueous phase drop wise and the mixture was homogenised at 20,000 rpm for 20 min low temperature. The emulsion was then simultaneously stirred (at 500 rpm) and sonicated in a bath sonicator for 1 hr. Gentle stirring using a magnetic stirrer for 24 hr evaporated the organic solvent. Finally, the nanoparticles were washed and concentrated using Centriprep concentrators at 3000× g for 2 hr. The process was repeated several times until there was no monensin in the washings.

3. Micro-particle

Principle Methods of Preparation:

For microparticles distinction is not made between spheres or capsules.

3.1 In situ polymerisation

3.2 Emulsification-evaporation and emulsification-extraction

3.3 Phase separation (coacervation)

3.4 Spray-drying (nebulisation) and spray coating (fluidisation)

3.5 Milling methods after cooling, compression or extrusion.

### EXAMPLE 7

Synthesis of a Microsphere (Chitosan)

Microspheres were prepared by adding citric acid, as a crosslinking agent, to 5 ml of an aqueous solution of chitosan. Chitosan aqueous acetic acid solutions were prepared at different percentages of chitosan (0.38%, 1%, 2%, 5%) maintaining constant molar ratio between chitosan and citric acid (6.90×10⁻³ mol chitosan:mol citric acid) and the same pH value as the aqueous preparative solution. The chitosan-crosslinker solution was frozen to 0° C. and added to 25 ml of corn oil at the same temperature, stirring for 2 min before adding to 75 ml of corn oil heated to 120° C. Thermal crosslinking was carried out for 40 min in a glass beaker under vigorous stirring (900 rpm) using a 4-bladed impeller (4-cm diameter). The microspheres obtained were separated by centrifugation, washed with 100 ml diethyl ether, dried and sieved. The fractions corresponding to a mean geometric diameter of 100 +/−10 mm were used.

4. Lipid Components

Cerebroside

Ethanolamine Phosphatides

Glycerolophosphoryl choline

4.1 Lecithins (Examples)

Bovine heart

Bovine spinal cord

Egg yolk

Soya Bean

Egg, hydrogenated

Lecithin mixtures

Lysolecithin

Lysophosphatidyl ethanolamine

Lysophosphatidly glycerol

Phosphatidic acid

Phosphatidyl butanol

Phosphatidyl ethanol

Phosphatidyl ethanolamine

Phosphatidyl glycerol

Phosphatidyl inositol

Phosphatidyl inostol 4,5, biphosphate

Phosphatidyl propanol

Phosphatidyl serine

4.2 Plant Leaf Lipids (Examples)

Digalactosyl diglyceride

Monogalactosyl diglyceride

Phospatidyl glycerol

Sulphoquinovosyl diglyceride

Sphingomyelin

Sulfaitde

Total lipid extract, Bovine spinal cord

4.3 Semi-synthetic Lipids (Examples)

Diacyl glycerol

Dilauroyl lecithin

Dilinoleoyl lecithin

Dimyristoyl lecithin

Dioctanoyl lecithin

Dioleoyl glycerol

Dioleoyl lecithin

Dioleoyl phosphatidyl ethanolamine

Dipalmitoyl lecithin

Dipalmitoyl phosphatidyl ethanolamine

Distearoyl lecithin

1-Lauroyl-2-lysolecithin

4.4 Chemical Classification-Lipid Classes (Examples)

Glycerophospholipid

Spingophospholipida

Glyceroglycolipid

Spingoglycolipid

4.4.1 Phospholipids-Examples

Symmetrical saturated Diacyl Glycerophospholipids

Symmetrical saturated Tetraacyl Diphosphatidylglycerol

Symmetrical unsaturated Diacyl Glycerophospholipids

Symmnetrical unsaturated Tetraacyl Diphosphatidylglycerol

Symmetrical saturated Dialkyl Glycerophospholipids

Symmetrical saturated Dialkyl Glycerophospholipids

Mixed-chain saturated Diacyl Glycerophospholipids

Saturated 1-Acyl-2-Acetyl Glycerophospholipids

Saturated/unsaturated mixed-chain Diacyl

Glycerophospholipids

Saturated 1-Alkyl-2-Acetyl Glycerophospholipids

Saturated 1-Acyl-2-Lyso Glycerophospholipids

Phosphatidylcholines

Lysophosphatidylcholines

Phosphatidylethanolamines

Lysophosphatidylethanolamines

Phosphatidylglycerols

Phosphatidic acids

Phosphatidylserines

Diphosphatidylglycerols (cardolipids)

Phosphatidylinositols

Di- and Triphosphoinositides

Sphingomyelins

4.4.2 Glycolipids-Examples

Glycoglycerolipids

Ceramides

Glycosphingolipids

Sialoglycosphingolipids

Glycosphingolipids

Cerebrosides

Glycosylsphingosines

Key Step 2: Attachment or coniugation of the support to the lipid laver or coating

Once the support has been synthesised, the lipid layer or coating must be attached. This can be effected by a variety of means. In the examples given the method used to attach the lipid layer or coating will vary. The reaction can be followed by several means including chromatography (GPC (SEC), HPLC or TLC) or gel electrophoresis.

For the dendrimers, whether synthesised by the divergent route or convergent route, the surface groups will effect the method used for attachment. If the dendrimer had a surface functional group such as an amine or carboxylate (e.g. examples 1, 2, 3, 5, 6, 7) then the water soluble carbodiimide 1-ethyl-3-(-dimethylaminopropyl) carbodiimide hydrochloride (EDC) was used to surface graft a lipid or coating with a functional group which is also an amine or carboxylate to form an amide bond. The same reaction could also be effected in an organic solvent using a carbodiimide such as dicyclohexyl carbodiimide (DCC, zero length coupler).

EDC conjugation where the dendrimer has a carboxylate (carboxylic acid) surface functionality and the lipid or coating has an amine

The dendrimer was dissolved in a suitable amount of water or buffer (PBS, phosphate buffered saline). The pH was adjusted to between 4-5.5 or just below neutral (pH of 6.5). The EDC was added slowly under stirring conditions at a molar ratio, which was equivalent to the amount needed to activate the all carboxy surface groups on the dendrimer. The intermediate was formed (activated EDC) relatively quickly (up to 30 mins, at room temperature). Then the lipid (the concentration of lipid was monitored when added so as to prevent the formation of micelles at or around the critical micelle concentration) or coating with the amine group was added to the dendrimer with activated carboxy groups. This then permitted the EDC to link the amine to the carboxy group and form a stable amide bond. The solution was then left to allow the reaction to go to completion (several hours, stirring). Unreacted EDC would hydrolyse to urea. The Articell™ was then purified by dialysis using a suitable membrane (Spectrpor), chromatography (gel permeation chromatography, ion exchange) or ultrafiltration using a suitable filter to allow the unreacted impurities to be removed.

HPLC, NMR (¹H, ¹³C, HCOSY, ¹³CCOSY), particle sizing (PCS) and mass spectrometry (MALDI-TOF, electron spray) were used to characterise the product.

EDC conjugation where the dendrimer has an amine surface functionality and the lipid or coating has a carboxylate (carboxylic acid)

The procedure used was similar to the previous one except the carboxy group on the lipid or coating was activated first using EDC and the amine terminated dendrimer was then added.

(In all EDC reactions the intermediate can be stabilised for longer periods by adding sulfo-NHS).

Although the association produced by electrostatic charge, hydrophobic interactions and hydrogen bonding, and schiff base intermediates are not as strong as a covalent bond, they can be useful should the need arise for the lipid layer or coating under certain conditions to be released. To allow the passage of molecules trapped within the cytoskeletal type of support to be released.

Preparation of support to lipid layer or coating using charge interactions

Where the support is charged the outer coating or lipid layer was attached by charge interactions. The two components were mixed and left to react at room temperature, under stirring conditions in an aqueous or non-polar solvent. After an hour or so dialysis, ultrafiltration or chromatography then purified the Articell™.

Preparation of support to lipid layer or coating using hydrophobic interactions (examples 4, 6, 7)

A quantity of dendrimer, nanoparticle or microparticle was dissolved in aqueous media (non-aqueous solutions can also be used). The lipid layer or coating was then applied by adding a quantity of the lipids to the solution. Because lipids are hydrophobic (or at least have a hydrophobic domain in the case of phospholipids), the hydrophobic lipids arrange themselves around the structural support to form a layer, in a similar way to the formation of a micellular structure. Purification of the Articell™ after formation of the structure was achieved by dialysis, ultrafiltration or chromatography.

Preparation of support to lipid layer or coating using hydrogen interactions

Where appropriate the lipid layer or coating was applied to the support on the basis of the formation of a hydrogen bond. Purification of the Articell™ after formation of the structure was achieved by dialysis, ultrafiltration or chromatography.

Preparation of support to lipid layer or coating using schiff base interactions

Where appropriate the lipid layer or coating was applied to the support on the basis of the formation of schiff base intermediates, which can be chemically stabilised by reduction (NaCNBH₃). Purification of the Articell™ after formation of the structure was achieved by dialysis, ultrafiltration or chromatography.

### POLYMER AGGREGATE AS SUPPORT

When synthesising a dendrimer according to step 1 (examples 1-5) and an aggregation effect is observed either due to whole generations, fragments or a combination of both forming such aggregates, the coating can be applied according to the methods described in step 2. Purification will yield an Articell™ with a polymeric aggregate as support.

### TUBULAR POLYMER AS SUPPORT

When the dendrimer is synthesised based on the methods according to step (examples 1-5) and a tubular type of structure is observed either as a result of a defect in branching causing the dendrimer to form such a structure during subsequent growth or when dendritic growth is restricted causing the formation of a tubular type structure; the coating applied according to step 2 will yield an Articell™ with a tubular type of support.

General note

In all the above cases, there is potential for entrapment in the pores or cavities of the support of therapeutic or bioactive molecules. These molecules can be dissolved in the solution during the stage at which the support is first dissolved, prior to the lipid or coating being applied. Hence on application of the coating the molecules will be trapped inside. Dialysis will remove untrapped or free molecules. This is in addition to the possibility of applying these molecules to, the surface of the Articell™. Release of trapped molecules could be effected by the support breaking up (e.g. ester linkages connecting a dendrimer core or branch units, triggered by a pH change) or the layer leaving the support (e.g. ester linkages between the support and coating, triggered by a pH change). Other linkages that could release the coating layer are thermodynamic, photosensitive and enzymatic sensitive linkages.

Other linkers that can be used for attachment of common end groups between the lipid layers or coatings and the support (some modification of groups may be required to obtain the desired group before conjugation)

Modification of amines with 2-Iminothiolane (Traut's reagent) to produce a sulfhydryl group

Modification of amines with SATA (N-succinimidyl S-acetylthioacetate) to introduce a sulfhydryl group

Modification of amines with SATP (succinimidyl acetyl-thiopropionate) as per SATA (protected sulfhydryl group).

Modification of aldehydes or ketones with AMBH (2-acetamido-4-mercaptobutyric acid hydrazide) to thiolate the aldehydes or ketones to produce sulfhydryl groups.

Modification of carboxylates or phosphates with cystamine to produce sulfhydryl groups.

EDC can be used in one or two step modifications of the following groups:

Sulfhydryls modified with ethylenimine or 2-bromoethylamine

Carbohydrates modified with diamines

Alkylphosphates with diamines

Aldehydes with ammonia or diamines

N,N′-Carbonyldiimidazole (CDI)

Activation of carboxylic acids or hydroxyl groups using CDI for conjugation to other nucleophiles using zero length amide bonds or one carbon length N-alkyl carbamate linkages

Other cross-linking reagents that can be used for coupling.

Carbodiimides

1-cyclohexyl-3-(2-morpholinoethyl) carbodiimide (CMC)

Dicyclohexyl carbodiimide (DCC)

Diisopropyl carbodiimide (DIC)

Examples of homofunctional cross-linkers

N-Hydroxysuccinimide (NHS)

Lomant's reagent [dithiobis (succinimidylpropionate)] (DSP)

Disuccinimidyl suberate (DSS)

Disuccinimidyl tartarate (DST)

Bis[2-(succinimidyloxycarbonyloxy)ethyl]sulfone (BSOCOES)

Ethylene glycolbis(succinimidylsuccinate) (EGS)

Disuccinimidyl glutarate (DSG)

N,N′-Disuccinimidyl carbonate (DSC)

Dimethyl adipimidate (DMA)

Dimethyl pimelimidate (DMP)

Dimethyl suberimidate (DMS)

Dimethyl 3,3′-dithiobispropionimidate (DTBP)

Formaldehyde

Glutaraldehyde

Bis epoxides

Adipic acid dihydrazide

Carbohydrazide

(And other similar Linkers)

Examples of heterobifuntional cross-linkers

N-Succinimidyl 3-(2-pyridyldithio)propionate (SPDP)

Succinimidyloxycarbonyl-a-methyl-a-(2-pyridyldithio)toluene (SMPT)

Succinimidyl-4-(N-maleimidomethyl)cyclohexane-1-carboxylate (SMCC)

m-Maleimidobenzoyl-N-hydroxysuccinimide ester (MBS)

4-(4-N-Maleimidophenyl)butyric acid hydrazide (MPBH)

(And other similar linkers (including heterotrifunctional))

In conclusion is should be noted that many of the classes of support structures detailed in the examples given, are commercially available for flirther modification. Therefore there is a great potential for future Articell™ development.

Whilst examples of the support structure of the invention and their production are given above, variations will be apparent to those skilled in the art which do not depart from the scope of the invention as defined in the appended claims. In particular, the invention also encompasses the support and its synthesis within a preformed vesicle or micelle. If the necessary components to begin a dendrimer synthesis reaction are added to a solvent or synthesis is already under way beyond generation 1, the addition of the coating components (lipids, cholesterol, or phospholipids) at a concentration above the critical micelle concentration (leading to the formation of a vesicle, micelle or liposomal type structure) would result in a proportion of the support being entrapped. Continued synthesis would allow the support to evolve or grow until it met the inner interface of the coating.

1. An intemally supported lipid vesicle system, the system comprising a branched polymeric structure which provides a structural support for a mono-layer, bi-layer or multi-layered lipid coating.

2. A system according to claim 1, wherein the structural support is a hyperbranched structure.

3. A system according to claim 1, wherein the structural support is a cascade polymer.

4. A system according to claim 1, wherein the structural support is an arborol.

5. A system according to claim 1, wherein the structural support is a dendrimer structure.

6. A system according to claim 1, wherein the structural support is a nanoparticle.

7. A system according to claim 1, wherein the structural support is a microparticle.

8. A system according to claim 1, wherein the structural support is a polymer.

9. A system according to claim 1, wherein the structural support is a tubular polymer.

10. A system according to claim 1, wherein the structural support is a polymeric aggregate.

11. A system according to any preceding claim, wherein the lipid coating layer is an anionic, cationic or neutral phospliolipid, the phospholipid being a glycerol ester.

12. A system according to any of claims 1 to 10, wherein the lipid coating layer contains a mixture of different percentages of anionic, cationic or neutral lipids, the lipid being a glycerol ester, esters of sphingol, cholesterol, glycolipids, or lipoproteins.

13. A system according to any of claims 1 to 10, wherein the coating layer is a reconstituted membrane of animal or plant cell, reconstituted bacterial membrane or viral capsid.

14. A system according to any preceding claim, wherein the coating layer additionally comprises natural or synthetic receptors or recognition sites.

15. A system according to any preceding claim, wherein the association between the structural support and the coating layer is a result of covalent, anionic, cationic, neutral. hydrogen bonding, hydrophobic or co-ordinate interaction.

16. A system according to any preceding claim, wherein there is a layer or chains of some other compound between the support and coating.

17. A system according to claim 16, wherein this layer of chains comprise carbohydrate, alkyl chains, fatty acids, amino acids, cholesterol, palmitoyl or derivatives thereof.

18. A system according to any preceding claim, wherein the system additionally comprises a pharmaceutically active agent.

19. A system according to claim 18, wherein the pharmaceutically active agent is reversibly associated with the structural support.

20. A system according to claim 18, wherein the pharmaceutically active agent is reversibly associated with the lipid coating.

21. A system according to any preceding claim, wherein a bioactive molecule is contained within the system and is releasable by a chemical, biochemical, thermal, pH, mechanical, electromagnetic trigger; by passing across the coating layer, through a conformational change or disruption of the layer(s).

22. A system according to any preceding claim, wherein the delivery route for administration is oral, nasal, intravenous, intraperitoneal, subcutaneous, pulmailary, intra-arterial, intramuscular, intracranial or transdermal.

23. A delivery system for the treatment or prophylaxis of disease, comprising a plurality of individual systems according to any of claims 1 to 21, contained within a larger, parent system and releasable from the parent by a chemical, biochemical, thermal, pH, mechanical, electromagnetic trigger; by passing across the coating layer, through a conformational change or disruption of the lipid layer.

24. A method for the production of a system according to any one of claims 1 to 22, wherein the synthesis of the support is initiated within a lipid coating that has been pre-formed in the form of a vesicle or liposomal structure, so that the branched structural support evolves or grows within the coating until its completion, the final structure being the support contained within the coating.

25. A method for the production of a system according to any of claims 1 to 23, wherein a branched polymer, dendrimer, arborol, star polymer, hyperbranched structure. cascade polymer or fragment thereof, such as a dendrimer branch or fragment synthesised by a convergent route, is assembled into a micelle structure, in an aqueous solvent, and then a lipid coating is applied.

26. A method according to claim 25, wherein the lipid coating layer is an anionic, cationic or neutral phospholipid, the phospholipid being a glycerol ester.

27. A method according to claim 25, wherein the lipid coating layer contains a mixture of different percentages of anionic, cationic or neutral lipids, the lipid being a glycerol ester, esters of sphingol, cholesterol, glycolipids, or lipoproteins.

28. A method according to any of claim 25, wherein the coating layer is a reconstituted membrane of animal or plant cell, reconstituted bacterial membrane or viral capsid.

29. A method of treatment or prevention of disease, comprising treating an animal or human with the system of any of claims 1 to 24.
